(19) United States
(12) Patent Application Publication
(10) Pub. No.: US 2010/0028406 A1
(43) Pub. Date: Feb. 4, 2010

(54) TOPICAL DRUG DELIVERY BY IONTOPHORESIS

(75) Inventors: Yogeshvar N. Kalia, Chevrier (FR); Aarti Naik, Chevrier (FR); Nada Abla, Geneva (CH)

Correspondence Address: FULBRIGHT & JAWORSKI L.L.P., 600 CONGRESS AVE., SUITE 2400, AUSTIN, TX, 78701, US

(73) Assignee: UNIVERSITE DE GENEVE, Geneve 4 (CH)

(21) Appl. No.: 11/997,525

(22) PCT Filed: Aug. 4, 2006
(86) PCT No.: PCT/IB06/04141

§ 371 Date: Aug. 28, 2008

Related U.S. Application Data
(60) Provisional application No. 60/705,929, filed on Aug. 5, 2005.
(60) Provisional application No. 60/760,978, filed on Jan. 20, 2006.

(51) Int. Cl. A61K 9/70; A61K 31/522; A61P 27/02

(52) U.S. Cl. 424/443; 514/263.3

(57) ABSTRACT

The invention generally concerns methods of topical drag delivery. Delivery according to the invention may be via electrotransport of compounds through the skin, for example by iontophoresis. In certain embodiments improved methods for the delivery of compounds, such as antimicrobial agents are described.

### BACKGROUND OF THE INVENTION

1. Field of the Invention

The invention generally relates to the field of topical drug delivery. Specifically the invention concerns the topical delivery of antimicrobial agents via iontophoresis.

2. Description of Related Art

The successful topical treatment of cutaneous herpes simplex virus (HSV) infections using acyclovir (ACV; FIG. 1) offers several advantages over systemic therapy: the drug can be directly targeted to its site of action, reducing circulating drug levels and hence, the attendant adverse effects. However, topical ACV creams, though extensively evaluated, have demonstrated only modest efficacy that is also partly dependent on a number of pathophysiological parameters, e.g., the type and phase of infection (primary vs recurrent; early vs latent), the severity of infection and the patient's immune status (Raborn and Grace, 2003; Spruance et al., 2002). Moreover, most studies investigating ointment formulations have shown little or no clinical benefit in the treatment of cutaneous lesions e.g., herpes labialis (Fiddian and Ivanyi, 1983; Spruance and Crumpacker, 1982; Spruance et al., 1984; Yeo and Fiddian, 1983). This vehicle effect has been attributed to the slightly improved permeation of ACV from creams relative to ointments as demonstrated across human (Freeman et al., 1986) and guinea pig (Spruance et al., 1986) skin in vitro. Although the inability of ACV to efficiently penetrate the stratum corneum (SC) barrier has been proposed as one of the principal reasons for inadequate topical ACV therapy (Freeman et al., 1986; Spruance et al., 2002), a dermatopharmacokinetic study has shown that while total epidermal concentrations of ACV subsequent to topical delivery are superior to those attained after oral administration, the latter appears to deliver more drug to the basal epidermis, the site of infection (Parry et al., 1992). Formulation strategies to enhance cutaneous ACV permeation have included the incorporation of enhancers such as dimethyl sulfoxide (Freeman et al., 1986; Spruance et al., 1984) and the use of polymeric vehicles (Piret et al., 2000).

Iontophoresis, the application of a small electrical current to facilitate the transport of charged molecules into and across the skin (Abla et al., 2006; Kalia et al., 2004), has also been investigated as a means to increase cutaneous ACV bioavailability (Gangarosa and Hill, 1995; Stagni et al., 2004; Volpato et al., 1995; Volpato et al., 1998). This technique enables the non-invasive, controlled administration of therapeutic agents for either local or systemic action, and has recently culminated in the commercialization of a device incorporating lidocaine for local anesthesia in infants prior to superficial dermatological procedures (LidoSite™, Vyteris, Inc., Fair Lawn, N.J.) (Kalia et al., 2004). The LidoSite™ device, in which the positively-charged lidocaine participates in the electrical circuit by transporting charge from the anodal compartment into the skin, results in local anaesthesia within 10 minutes—within which period the drug reaches the nerves located in the dermis and epidermis.

Although the delivery of therapeutic amounts of ACV into human skin in vitro after 30 minutes of current application followed by 5 hours of passive delivery has been reported (albeit with a formulation pH of 3) (Volpato et al., 1998), ACV is not an ideal candidate for topical iontophoresis as it is essentially uncharged at physiological pH (pKₐ₁ 2.27; pKₐ₂ 9.25) and has a low aqueous solubility (1.3 mg/mL at pH 7.4, 25°). In comparison, valaciclovir (VCV; FIG. 1), the L-valyl ester prodrug of ACV (Beauchamp et al., 1992; Jacobson, 1993) possesses three ionizable groups with pKa values of 1.90, 7.47 and 9.43. Consequently, VCV is ˜50% protonated at physiological pH, and more suited to iontophoresis relative to its active metabolite, ACV. VCV is rapidly and extensively converted to ACV by intestinal and/or first pass hepatic metabolism subsequent to oral administration (Anand et al., 2004; Soul-Lawton et al., 1995). Although, to-date, VCV has not been used topically, the considerable esterase activity within the skin (Boehnlein et al., 1994; Martin and Axelrod, 1957) and observed hydrolysis of peptide linkages after transit through the lipidic SC (the rate-determining barrier to cutaneous transport of polar and charged molecules, (Abla et al., 2005), together with the physical chemistry of VCV, suggest that this prodrug may prove to be a model candidate for cutaneous iontophoresis.

The iontophoresis of ester prodrugs, in particular lipophilic alkyl ester prodrugs, has been the subject of several studies (Gerscher et al., 2001; Gerscher et al., 2000; Lopez et al., 2003; Stinchcomb et al., 1996; Sung et al., 2000). However, the use of an amino acid ester prodrug to augment the charged nature of a drug, and thus its iontophoretic permeation, has not been studied in detail. The concept has been exploited for delivering dehydroepiandrosterone (DHEA); iontophoresis of the ionized glycyl ester of DHEA led to a modest 3-fold increase in permeation through rabbit skin, compared to the parent DHEA (Laneri et al., 1999).

The aim of this study was to investigate the iontophoretic delivery of VCV, exploiting its positive charge to facilitate electrotransport across the skin and to rely on cutaneous esterase activity to release increased amounts of ACV at or near the site of action. The enhanced delivery of ACV to the basal epidermis, via the use of a substantially charged prodrug, would thus be expected to ameliorate the treatment of cutaneous herpetic infections by targeting therapeutic levels of drug to the site of action without the undue systemic exposure associated with oral and parenteral delivery. Importantly, these studies demonstrate the efficient topical delivery of charged antimicrobial agents. Thus, the present study addresses a long standing need in the art by providing methods for the efficient electrotransport of topically applied antimicrobial compounds.

### SUMMARY OF THE INVENTION

In certain embodiments, methods are provided for topical delivery of a charged antimicrobial compound to an animal. The method involves a composition comprising molecules of an antimicrobial compound, wherein more than 20% of the molecules would be charged at pH 7.4. The composition is applied to an affected area of an animal, and the affected area is subjected to an electrical current in a manner effective to promote the transport of the compound in the skin of said animal. In some embodiments, antimicrobial compositions comprise antimicrobial molecules wherein more than about 30%, 40%, 50%, 60%, 70%, 80%, 90% or 95% of the molecules would be charged at pH 7.4. As used herein the term charged refers to either a positive or negative charge of 1 or greater.

In certain embodiments, methods are provided for topical delivery of a protonated antimicrobial compound to an animal. The method involves a composition comprising molecules of an antimicrobial compound, wherein more than 20% of the molecules would be protonated at pH 7.4. The composition is applied to an affected area of an animal, and the affected area is subjected to an electrical current in a manner effective to promote the transport of the compound in the skin of said animal. In some embodiments, antimicrobial compositions comprise antimicrobial molecules wherein more than about 30%, 40%, 50%, 60%, 70%, 80%, 90% or 95% of the molecules would be protonated at pH 7.4. In yet further embodiments, the method involves a composition comprising molecules of an antimicrobial compound, wherein more than 20% of the molecules would be diprotonated at pH 7.4. Furthermore, in some cases, antimicrobial compositions comprise antimicrobial molecules wherein more than about 30%, 40%, 50%, 60%, 70%, 80%, 90% or 95% of the molecules would be diprotonated at pH 7.4. In yet a further embodiment an antimicrobial compound of the present invention will have substantial aqueous solubility. For example, an antimicrobial compound according to the invention may have a solubility of greater than about 1.3 mg/ml, 1.5 mg/ml, 2.0 mg/ml or 2.5 mg/ml.

In certain embodiments, antimicrobial compounds of the invention may comprise an amino acid ester group, a peptide bond or an ester. In some cases antimicrobial compounds are in an active form in the composition, however in other cases the antimicrobial formulations may be pro-drugs that are converted to their active form by mixing with a second agent, or by cellular or extra cellular enzymes. As used herein the term antimicrobial compound comprises antiviral, antifungal, antibiotic, bacteriostatic agents, and refers to both their active and pro-drug forms. In some cases antibiotic according to the composition may be trimethoprim, vancomycin, erythromycin, clairithromycin, oleandomycin, troleandomycin, or a quinolone such as norfloxacin, enoxacin, ciprofloxacin, ofloxacin, levofloxacin, lemefloxacin, gatifloxacin, trovafloxacin, sparfloxacin, or moxifloxacin. In certain cases, amino acid esters macrolid antibiotics may also be used, such as erythromycin, clairithromycin, oleandomycin, or troleandomycin amino acid esters. In another specific embodiment an antimicrobial compound according to the invention may be an antifungal compound, such as ketoconazole. Furthermore, in certain embodiments an antimicrobial compound according to the invention may be an amino acid ester of an antiviral compound such as penciclovir, ganciclovir, 6-deoxyaciclovir, cytarabine, vidarabine, idoxuridine, trufluorothymidine, ribavirin, zidovudine, didanosine, zalcitabine, stavudine, lamivudine, or abacavir amino acid ester. In some specific embodiments antiviral compounds may be herpes antiviral compounds such as valaciclovir. Antimicrobial compositions according to the invention maybe for systemic or local delivery.

In certain cases, antimicrobial compositions will additionally comprise a buffer system. For example, a phosphate, carbonate, HEPES or TRIS buffer system. Thus, in some embodiments, the pH of the antimicrobial compositions is about, at least about or less than about 4.0, 4.1, 4.2, 4.3, 4.4, 4.5, 4.6, 4.7, 4.8, 4.9, 5.0, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0 or any range derivable therein. In certain aspects of the invention, the antimicrobial composition has a pH of between about 6.0 and 8.0 or about 6.5 and 7.8 or about 7.0 and 7.5. In certain specific aspects, antimicrobial composition have a pH near a physiological pH of 7.4. In some aspects of the invention the pH of the composition is adjusted to a range at which the largest portion of the antimicrobial molecule is charged or protonated.

In some embodiments antimicrobial compositions comprise additional agents, for example moisturizers, salts, preservatives and/or anesthetics. For example, anesthetics such as lidocaine, bupivacaine, butacaine, chloroprocaine, cinchocaine, etidocaine, mepivacaine, prilocaine, ropivacaine and/or tetracaine may be included, and may lessen the discomfort associated with use of electrical current.

In further aspects of the invention, antimicrobial compositions according to the invention are provided as patches. In certain cases the patches may comprise water soluble polymers, furthermore patches according to the invention may be clear or transparent. It will be understood by the skilled artisan that patches comprising a water soluble cross-linked polymer (e.g., as the drug reservoir) will additionally comprise a non-water soluble backing. For example, a patch of the invention may be part of a “split” system, that is, a reusable device plus a disposable patch comprising the drug in such a water soluble polymeric drug reservoir. Thus, the latter may be placed in contact with the skin while the insoluble backing may be used to house an iontophoresis electrode and/or protect the contents of the patch from the environment. Similarly, in an integrated system, a patch backing may also isolate the drug reservoir from the electronic components in the controller (which may also have its own “housing”). Patches may be in a variety of sizes and shapes. For example patches may cover an area of less than about 100 cm², less than about 10 cm² or less than about 5 cm². In certain embodiments, patches according to the invention may be applied to the eye.

In certain embodiments, antimicrobial compositions according to the invention may be applied to an affected topical area of an animal. In some aspects, the animal may be a human. In certain cases, the affected topical area is an eye, lips, face skin, genitals or anus. In certain cases the topical area is a lesion, for example a viral, fungal, or bacterial lesion. In certain specific aspects, the lesion is a herpes viral lesion, such as a Herpes simplex virus (HSV)-1, HSV-2 or Varicella Zoster Virus (VZV) lesion. As used herein the term lesion can mean a rash, sore, cut, or otherwise inflamed area. Such lesions may be in any topical area including but not limited to the torso, the arms and legs, the face, the eyes, the genitals, and the anus. Thus, certain aspects the invention involve treatment for ocular, genital, anal, or facial herpes viral lesions. The area may be of essentially any size and shape for example the area may be less that about 10 cm² or less than about 5 cm².

In certain aspects of the invention, an electrical current is applied to an affected topical area following the application of an antimicrobial composition. In certain cases, a current density of less than about 0.5 mA/cm² may be applied. In some cases, a current density of less than about 0.4 mA/cm², or less than about 0.375 mA/cm² may also be used. Thus in certain aspects of the invention, a current density of about 0.01 to about 0.5 mA/cm², or about 0.05 to about 0.2 mA/cm² is used. In some cases, the electrical current is applied with an iontophoresis apparatus. Examples of iontophoretic devices include but are not limited to, the Phoresor II Auto, the Phores PM900 and the Empi Dupel. In some case the current may be applied for a period of time. For example for 4 hours or less, or for one hour or less.

In certain aspects, antimicrobial compositions according to the current invention are comprised in a reservoir. In some cases a reservoir will be part of an electrode assembly, however in any case it will be comprised of an electrically conductive material that may placed in contact with an electrode. Such reservoirs enable the delivery of at least one medicament through an applied area of a patient, such as the skin, or mucous membrane. In some cases, reservoirs according to the invention comprise a layer of an aqueous swollen cross linked water soluble polymer material capable of having electrocontinuity with the electrode assembly. In further embodiments, the aqueous swollen cross linked water soluble polymer material has sufficient adhesive tack for adherence to a surface. In yet further embodiments, the aqueous swollen cross linked water soluble polymer material has an adhesive strength to the electrode material greater than the cohesive strength of the polymer material, and the cohesive strength is greater than an adhesive strength to the applied area.

In some embodiments, a reservoir according to the invention also includes a structurally reinforcing member situated within the layer of aqueous swollen cross linked water soluble polymer material. For example, a structurally reinforcing member may have approximately 40% porosity so as not to impede the flow of ions. In some aspects the structural reinforcing member is a wettable, scrim of an aqueous insoluble thermoplastic polymeric material. In some specific cases, the aqueous swollen cross linked water soluble polymer material is cross linked by high energy irradiation. In yet a further embodiment, the aqueous swollen cross linked water soluble polymer is polyethylene oxide, polyvinyl pyrrolidone, polyvinyl alcohol, polyacrylamide or polyethylene glycol. In some specific embodiments, a reservoir additionally comprises a vasoconstrictor, a stabilizer and/or glycerin. Further, a reservoir may comprise additives and conductive salts. For example, glycerin, propylene glycol, polyethylene glycol and/or preservatives may be comprised in a reservoir.

Kits are also provided as part of the present invention. In certain embodiments, kits comprise a composition comprising molecules of an antimicrobial compound wherein more than 20% of the molecules would be charged at pH 7.4 in addition to an apparatus capable of supplying an electrical current in a manner effective to transport the compound through the skin. In certain embodiments, kits comprise a composition comprising molecules of an antimicrobial compound wherein more than 20% of the molecules would be protonated at pH 7.4 in addition to an apparatus capable of supplying an electrical current in a manner effective to transport the compound through the skin. In some cases, kits according to the invention will include additional elements. For example, kits may include instructions for its use, and wires or electrodes, that act as anode and cathode. In some cases, the kit will be comprised in a box. In certain specific embodiments, the antimicrobial composition in the kit may be supplied as a patch, however it may also be supplied in a sealed container such as a syringe. Therefore, in certain cases, kits according to the invention may also include a patch, for example a patch to which the antimicrobial composition may be applied. It will be understood by one of skill in the art that kits according to the current invention may further include elements allowing the practice of the methods described above.

It will be understood that the invention also concerns methods for treating a topical lesion in an animal. Such a method comprises obtaining an antimicrobial composition according to the invention, applying the composition to an affected area of the animal, and subjecting the affected topical area to an electrical current in a manner effective to promote the transport of the compound through or into the skin of said animal. Topical lesions for treatment according to the invention include, but are not limited to, herpes viral lesions, lesions associated with leprosy, syphilitic lesions, acne, athletes foot, shingles, and warts.

In some embodiments, the invention also concerns methods for treating ocular infections in an animal. Such a method comprises obtaining an antimicrobial composition according to the invention, applying the composition to a topical area of the animal, and subjecting the affected topical area to an electrical current in a manner effective to promote the transport of the compound through or into the skin of said animal. In certain specific embodiments, methods according to the invention may be useful in treating optical infections, such as viral, bacterial or fungal infections. For example, conditions such as herpes keratoconjuctivitis or keratitis may be treated by methods according to the inventions. Such treatments may comprise iontophoretic delivery of trifuridine, vidarabine and/or idoxuridine.

In some further cases, methods according to the invention may be used to treat CMV-related infections of the eye(s) such as CMV retinitis. Thus, in certain cases methods for treating ocular CMV infections may involve applying compositions and methods according the invention directly to the cornea or conjunctiva. For example, such methods may comprise iontophoretic delivery of foscarnet, cidofovir, formivirsen, ganciclovir and/or valganciclovir. In certain preferred embodiments, CMV retinitis may be treated by iontophoretic delivery of ganciclovir or valganciclovir.

Embodiments discussed in the context of a methods and/or kits of the invention may be employed with respect to any other method or composition described herein. Thus, an embodiment pertaining to one method or kit may be applied to other methods and kits of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawing is part of the present specification and is included to further demonstrate certain aspects of the present invention. The invention may be better understood by reference to the drawing in combination with the detailed description of specific embodiments presented herein.

FIG. 1: Structure of VCV, the bracketed portion to the structure is ACV.

FIG. 2: A representative in vitro iontophoresis apparatus.

FIG. 3: Receptor levels of ACV after iontophoresis of ACV (2 mM) and VCV (2 and 10 mM) at 0.5 mA/cm² across porcine skin in vitro.

FIG. 4: The influence of Na⁺ ions in the 10 mM VCV formulation on VCV flux across the skin (estimated from ACV levels in the receptor compartment).

FIG. 5: Acetaminophen flux reported on the effect of VCV iontophoresis on skin permselectivity.

FIG. 6: Effect of formulation conditions on the contributions of EM and EO to the steady state iontophoretic flux of VCV across porcine skin in vitro.

### DETAILED DESCRIPTION OF THE INVENTION

In general, the rate and extent of drug permeation across a biological membrane depends on the physicochemical properties of the molecule. It must possess the correct balance of lipophilicity and hydrophilicity to enable its entry, transit and exit from these essentially lipidic membrane barriers. Passive permeation is governed by the concentration gradient across the rate-limiting barrier to transport, it is subject to Fick's Laws of Diffusion which state that at steady state the flux across the membrane depends upon the drug diffusion coefficient (or diffusivity). This parameter is inversely related to molecular volume which, in many mathematical models, is frequently approximated by the molecular weight. Thus, increasing molecular weight tends to reduce diffusivity and decrease transport. Moreover, introducing charge into a molecule may be expected to decrease its membrane permeation given the lipidic nature of the barrier. Therefore, it is counter-intuitive that increasing the molecular weight and/or charge of an antimicrobial compound would increase the ability of the compound diffuse to access deeper tissue layers, that are often the location of active microbial infections. However, in the example cited here, the molecular weight of valaciclovir (MW 325.5) is significantly higher than that of aciclovir (MW 225.2 Da) and yet its transport has been demonstrated to be more than 200-fold greater. Thus, introduction of a charged moiety in valaciclovir is shown to more than compensate for the increase in the molecular weight and facilitates its delivery across the skin via iontophoresis. This is of particular advantage in the case of herpes viral infections since the cells in which the virus actively replicates (neuronal cells) are well below the topical area where the antiviral is applied.

Thus, in some embodiments a method according to the invention involves delivery of antimicrobial compounds wherein a significant portion of the molecules are charged at physiological pH. Delivery of the antimicrobial agents can be via iontophoresis. In this respect these methods have significant advantages over other delivery methods. Studies undertaken by the inventors show that amino acid ester pro-drugs such as VCV are stable in solution, but rapidly and efficiently converted to an active form by esterase activity in the dermis. Thus, amino acid ester pro-drugs may be efficiently delivered to tissue via iontophoresis, and then are rapidly converted to their active forms by cellular and extracellular enzymes.

Previous studies have shown that the iontophoretic administration of ACV across human skin results in epidermal concentrations (˜80 μg/cm³), which are well above those required for viral inhibition (Volpato et al., 1998). Nevertheless, since iontophoretic efficiency is dependent on the electrical mobility of the drug, the electrotransport of charged species is clearly favored; for ACV, this is only possible in relatively acidic formulations (pKa=2.27). In contrast, the dissociation profile of VCV is much more conducive to iontophoretic delivery, allowing anodal delivery from formulations at physiological pH. Indeed, our results show that VCV iontophoresis for only 3 hours is almost 200-times more efficient compared to iontophoretic ACV delivery. In view of the comparable ACV transport across porcine skin in the present investigation and that previously observed across human skin, which produced therapeutic levels within the skin, the therapeutic superiority of VCV iontophoresis is evident. As with ACV, a further advantage offered by the electrically-mediated delivery of the prodrug is the rapidity with which steady-state kinetics are achieved, permitting short application times.

Given the iontophoretic transport efficiency of VCV (transport number ˜0.03, calculated from J_{EM} after iontophoresis for 7 hours), it is clear that clinically efficacious dosing is likely to be achieved with significantly reduced doses and/or more innocuous currents. For example, in view of the results here, a 10-fold reduction in applied current density (to 0.05 mA/cm², a value 10-fold lower than the so-called “limit of tolerability”) is still likely to achieve skin concentrations well above the therapeutic levels previously reported.

With respect to topical applications, the presence of esterase and peptidase activity (principally) in the viable skin layers and, therefore, after passage across the rate-limiting stratum corneum, (Boderke et al., 1997; Potts et al., 1989; Steinstrasser and Merkle, 1995; Abla et al., 2005) opens the door for many iontophoretic applications using prodrugs comprising peptide or ester bonds which are susceptible to enzymatic hydrolysis.

The present study demonstrates, unequivocally, the superiority of VCV iontophoresis over that of the parent molecule, ACV, to deliver the latter to the skin tissues. In doing so, it illustrates the pivotal role of drug physical chemistry in topical iontophoretic transport and serves to emphasize the impact of drug design on therapeutic efficiency. Though drugs are rarely custom-designed for the chosen route of administration, it is clear that chemical modifications can dramatically alter their transport behavior across a given biological membrane.

I. Additional Components

Antimicrobial compositions of the present invention can include other beneficial agents and compounds such as, for example, acute or chronic moisturizing agents (including, e.g., humectants, occlusive agents, and agents that affect the natural moisturization mechanisms of the skin), anti-oxidants, emollients, anti-irritants, vitamins, trace metals, anti-microbial agents, botanical extracts, salts, buffering agents, fragrances, and/or dyes, color ingredients and skin coolants, such as ethyl chloride (chloroethane) and/or fluori-methane.

### Salts and Buffers

Some nonlimiting examples of salts that may be used in accordance with the current invention include sodium chloride, magnesium chloride, magnesium sulfate, or any other substantially conductive salt.

Antimicrobial compositions according the current invention may also comprise a buffer or mixtures of buffers for example any of the buffer listed in

TABLE 1

| pH range | pKa | Buffer |
|---|---|---|
| 4.0-6.0 | 5.13 | malate (pK2) |
| 4.9-5.9 | 5.23 | pyridine |
| 5.0-6.0 | 5.33 | piperazine (pK1) |
| 5.0-7.4 | 6.27 | cacodylate |
| 5.5-6.5 | 5.64 | succinate (pK2) |
| 5.5-6.7 | 6.10 | MES |
| 5.5-7.2 | 6.40 | citrate (pK3) |
| 5.5-7.2 | 6.24 | maleate (pK2) |
| 5.5-7.4 | 1.70, 6.04, 9.09 | histidine |
| 5.8-7.2 | 6.46 | bis-tris |
| 5.8-8.0 | 7.20 | phosphate (pK2) |
| 6.0-12.0 | 9.50 | ethanolamine |
| 6.0-7.2 | 6.59 | ADA |
| 6.0-8.0 | 6.35 | carbonate (pK1) |
| 6.1-7.5 | 6.78 | ACES |
| 6.1-7.5 | 6.76 | PIPES |
| 6.2-7.6 | 6.87 | MOPSO |
| 6.2-7.8 | 6.95 | imidazole |
| 6.3-9.5 | 6.80, 9.00 | BIS-TRIS propane |
| 6.4-7.8 | 7.09 | BES |
| 6.5-7.9 | 7.14 | MOPS |
| 6.8-8.2 | 7.48 | HEPES |
| 6.8-8.2 | 7.40 | TES |
| 6.9-8.3 | 7.60 | MOBS |
| 7.0-8.2 | 7.52 | DIPSO |
| 7.0-8.2 | 7.61 | TAPSO |
| 7.0-8.3 | 7.76 | triethanolamine (TEA) |
| 7.0-9.0 | 0.91, 2.10, 6.70, 9.32 | pyrophosphate |
| 7.1-8.5 | 7.85 | HEPPSO |
| 7.2-8.5 | 7.78 | POPSO |
| 7.4-8.8 | 8.05 | tricine |
| 7.5-10.0 | 8.10 | hydrazine |
| 7.5-8.9 | 8.25 | glycylglycine (pK2) |
| 7.5-9.0 | 8.06 | Trizma (tris) |
| 7.6-8.6 | 8.00 | EPPS, HEPPS |
| 7.6-9.0 | 8.26 | BICINE |
| 7.6-9.0 | 8.30 | HEPBS |
| 7.7-9.1 | 8.40 | TAPS |
| 7.8-9.7 | 8.80 | 2-amino-2-methyl-1,3- |
| | | propanediol (AMPD) |
| 8.2-9.6 | 8.90 | TABS |
| 8.3-9.7 | 9.00 | AMPSO |
| 8.4-9.6 | 9.06 | taurine (AES) |
| 8.5-10.2 | 9.23, 12.74, 13.80 | borate |
| 8.6-10.0 | 9.50 | CHES |

### Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid; sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

### Antioxidants

Non-limiting examples of antioxidants that can be used with the compositions of the present invention include acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

### Preservatives

Non-limiting examples of preservatives that may used with compositions of the invention include Phenonip™, and/or any of its constituents phenoxyethanol, methylparaben, butylparaben, ethylparaben, propylparaben, additionally Suttocide®, Germaben™, LiquiPar potassium sorbate, and/or rosemary oleoresin may be used.

### Additional Compounds and Agents

Non-limiting examples of additional compounds and agents that can be used with the compositions of the present invention include, vitamins (e.g. D, E, A, K, and C), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), dyes and color ingredients (e.g. D&C blue no. 4, D&C green no. 5, D&C orange no. 4, D&C red no. 17, D&C red no. 33, D&C violet no. 2, D&C yellow no. 10, D&C yellow no. 11 and DEA-cetyl phosphate), emollients (i.e. organic esters, fatty acids, lanolin and its derivatives, plant and animal oils and fats, and di- and triglycerides), antimicrobial agents (e.g., triclosan and ethanol), and fragrances (natural and artificial).

II. Iontophoresis Devices

Some examples of iontophoresis devices that may be used according to the current invention include, but are not limited to, the Phoresor II Auto, the Phores PM900, the Empi Dupel, the apparatuses described in U.S. patent applications 20050113738, 20050070840, 20040167460, 20040116964, 20040064084, 20040039328, 20030135150 and U.S. Pat. Nos. 6,731,987 and 6,064,908.

In certain embodiments, iontophoressis devices for use according to the invention may comprise an apparatus that is carried on the body during treatment. For example, the apparatus man be worn in clothing or adhered to a portion of the body. Thus, in certain aspects the apparatus may comprise a power source that is placed at distance from the treatment site, but connected via to the site via wires or an interconnect. For example, the LidoSite™ iontophoresis device available from Vyteris, Inc.® (www.vyteris.com). In the case where the antimicrobial compositions are applied to the eyes, the wires for the apparatus may be supported, for example by eyeglasses.

III. Formulations for Antimicrobial Compositions

Antimicrobial compositions according to the current invention may be provided in a variety of formulations. For example as liquids, creams, salves, ointments, gels, or eye drops. In certain cases antimicrobial compositions may be comprised in a reservoir. In some cases such reservoirs may comprise patches, for example hydrogel patches. However it will be understood by one of skill in the art that an important characteristic of an antimicrobial composition, and of a reservoir comprising said antimicrobial composition to that it is able to conduct electricity. Some examples of polymers that may comprise a reservoir or patch include but are not limed to aqueous swollen cross linked water soluble polymers such as polyethylene oxide, polyvinyl pyrrolidone, polyvinyl alcohol, polyacrylamide or polyethylene glycol. Additional composition for reservoirs are described in detail in U.S. Pat. Nos. 6,862,473, 6,858,018, 6,629,968, 6,377,847, 5,882,677, and 5,738,647, all incorporated herein by reference. Other examples of patch design are described in U.S. Patent Applications 20030175328 or 20030175333 or PCT publication WO 2004062600.

In certain embodiments, it is contemplated that an antimicrobial composition may be applied separately from the electrode of the iontophoresis apparatus. For example, in certain embodiments the antimicrobial composition may be topically applied followed by application of the iontophoresis electrode. In certain cases the antimicrobial compostions may be applied multiple times to the electrode area during iontophosis to enhance the efficacy of the treatment.

For ocular applications the antimicrobial may be formulated as an eye drop and the iontophesis electrode may be a patch that can be applied to the eye. In this case the antimicrobial agent may be applied multiple times during the iontophoresis process to enhance transport of the antimicrobial drug or precursor. Alternatively, or in addition, an antimicrobial composition according to the invention may be incorporated into a patch that is to be applied to the eyes. Thus, in certain specific embodiments the iontophoresis electrode may be formed in to or connected to a patch that is in the shape of the eye surface, similar to a contact lens. In certain specific cases, it is contemplated that the electrode may be formed such that it can act as a prescription contact lens.

IV. Kits

In further embodiments of the invention, there is provided a kit. Any of the compositions, compounds, agents, or active ingredients described in this specification may be comprised in a kit, as described above. In a non-limiting example, a kit can include a composition comprising molecules of an antimicrobial compound according to the invention, in addition to an apparatus capable of supplying an electrical current in a manner effective to transport the compound through the skin.

The container means of the kits can include a bottle, dispenser, package, compartment, or other container means, into which a component may be placed. Where there is more than one component in the kit (they may be packaged together), the kit also will generally contain a second, third or other additional containers into which the additional components may be separately placed. The kits of the present invention also can include a means for containing the components in close confinement for commercial sale. Such containers may include injection or blow-molded plastic containers into which the desired bottles, dispensers, or packages are retained. For example, a kit of the present invention may include a container that has at least 2, 3, 4, 5, or more separated compartments. One compartment may include antimicrobial compositions while the other compartment includes an iontophoresis apparatus.

A kit can also include instructions for employing the kit components as well the use of any other compositions, compounds, agents, active ingredients, or objects not included in the kit. Instructions may include variations that can be implemented. The instructions can include an explanation of how to apply, use, and maintain the products or compositions, for example.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials Used in Studies

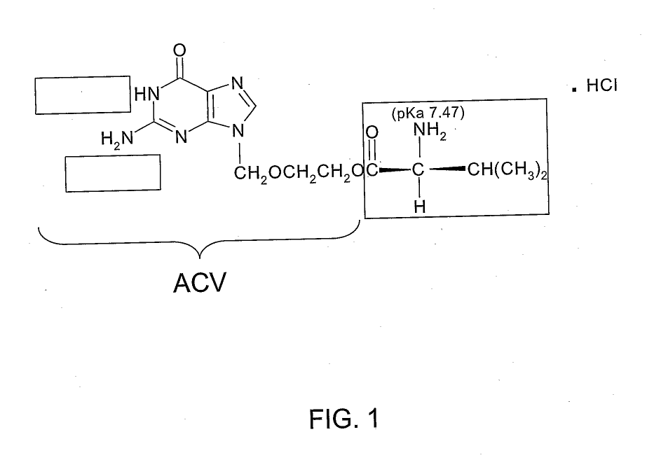

ACV and MeCN (Acetonitrile Chromasolv® for HPLC, gradient grade) were purchased from Sigma-Aldrich (Saint Quentin Fallavier, France). Acetaminophen, sodium chloride (NaCl), di-sodium hydrogen phosphate (Na₂HPO₄), potassium dihydrogen phosphate (KH₂PO₄), and trifluoroacetic acid (TFA) were purchased from Fluka (Saint Quentin Fallavier, France). VCV (see FIG. 1) HCl (99.5% purity) was purchased from Sequoia Research Products (Oxford, United Kingdom). All the solutions were prepared using de-ionized water (resistivity >18 MOhm cm).

Porcine ear skin, which is a well-accepted model for human skin (Dick and Scott, 1992; Sekkat et al., 2002), was used in these studies. Porcine ears were obtained from a local abattoir (Société d'Exploitation d'Abbatage, Annecy, France) a few hours after the sacrifice of the animals. The excised skin was then dermatomed (˜750 μm) on the same day and stored at −20° C. for a maximum of two months.

### Example 2

### Iontophoresis

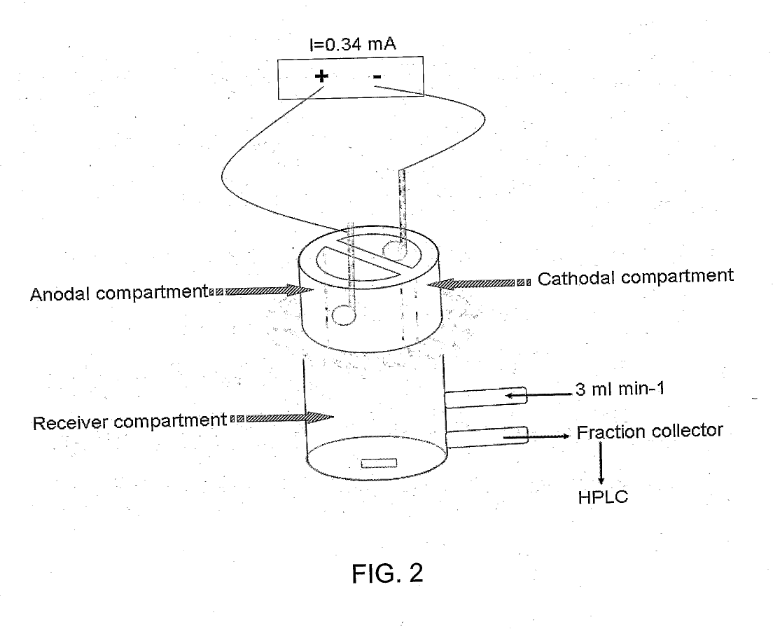

Iontophoresis was performed using vertical 3-compartment cells (FIG. 2). The skin was placed between two half-cells: the upper half, in contact with the SC, comprised two electrode/donor compartments, while the lower receiver compartment was in contact with the dermis. A flow-through system circulated phosphate-buffered normal saline (PBS: 16.8 mM Na₂HPO₄, 1.4 mM KH₂PO₄ and 136.9 mM NaCl; pH 7.4) through the receiver chamber (volume 4.7 mL) at a rate of 3 mL/h. Ag/AgCl electrodes were used throughout the study. The skin was allowed to equilibrate for one hour prior to the iontophoresis experiment. In order to reduce competition from Na⁺ ions present in the donor, and thus to increase the permeation of VCV, most experiments were performed using a salt bridge assembly. This strategy consists of physically separating the anodal chamber (Ag electrode immersed in PBS pH 7.4) from the donor compartment (drug solution in contact with the SC) and employing a salt bridge (prepared by filling a 12 cm tubing with a warm aqueous solution of 3% agarose and 0.1 M NaCl and then allowed to cool) to electrically connect the two chambers. The donor contained either 2 or 10 mM VCV.HCl in water, or 2 mM ACV in 2 mM NaCl: in the case of ACV, Cl-ions were provided by extraneous NaCl. For control experiments without the salt bridge, the anodal compartment contained 1 mL of 10 mM VCV.HCl in PBS pH 7.4. The cathodal compartment contained PBS pH 7.4 in all the experiments. A constant current of 0.34 mA was applied for 7 hours (equivalent to a current density of 0.5 mA/cm²). For passive controls, the donor contained 1 mL of 10 mM VCV.HCl in water or 2 mM ACV in 2 mM NaCl.

Acetaminophen (ACE, 15 mM) was included in the donor solution as a marker for electroosmotic flow. Being a small, polar and neutral molecule, it is transported mainly by convective solvent flow during iontophoresis. Therefore, its flux can be used to determine the contribution of electroosmosis to the total iontophoretic flux,

J_{EO}=vA→C*C_{D} (equation 1)

where J_{EO} represents the contribution of electroosmosis (EO) to delivery, v_{A→C} is the convective solvent flow from the anode to cathode, and C_{D} is the donor concentration of the drug.

The experiments were performed in sextuplicate, except for passive delivery (n=3), using the skin of as many different animals as possible. Samples were collected hourly and analyzed by high pressure liquid chromatography (HPLC). The HPLC system consisted of a 600 E Controller pump, an Autosampler Injector 717-plus, an In Line Degasser, and a UV 2487 dual λ Detector (Waters, Saint-Quentin Yvelines, France). The separation was performed on a Nucleosil® 100-5 C18 Nautilus column (L: 125 mm; ID: 4.6 mm; Macherey-Nagel, Hoerdt, France) with a 99:1 mixture of 0.1% trifluoroacetic acid (TFA) in water (pH 2.5): MeCN. The flow rate was 1 mL/min, the temperature was adjusted to 30° C., and 50 μL of sample were injected. ACV, ACE and VCV were separated after 5.6, 14.4 and 19.1 minutes, respectively, and detected at 252 nm (ACV and VCV) and 243 nm (ACE), enabling simultaneous analysis of the prodrug, drug and electroosmotic marker. The limits of detection/quantification for ACV, VCV and ACE were approximately 0.1/0.3, 0.25/0.8, and 0.2/0.6 μM, respectively.

### Example 3

### VCV Stability

The aqueous stability of VCV in the donor formulations was investigated by periodic sampling of solutions (2 and 10 mM in water with 15 mM acetaminophen) over a period of 44 hours. In addition, the impact of an electrical current (0.34 mA) on this stability was examined over the same period. The chemical hydrolysis of VCV was also investigated in the presence of PBS pH 7.4. The cutaneous conversion of VCV to ACV via hydrolytic cleavage of the ester bond was verified as follows. Cells were assembled as for an iontophoresis experiment. A 100 μM solution of VCV in PBS pH 7.4 was placed in all the cell compartments and left in contact with the skin (SC and dermis) for 4 hours. The compartments were assayed for ACV and VCV immediately afterwards. Finally, the same experiment was performed with a 100 μM solution of ACV, to examine the stability of ACV when in contact with the skin.

Unbuffered aqueous solutions of VCV (2 mM: pH 5.65; 10 mM: pH 5.24) were very stable in the presence of acetaminophen for the duration of the investigation (44 hours). Less than 1% of the VCV was converted into ACV over this time-period in the absence of a current; the extent of degradation was slightly increased in the presence of a current but nevertheless represented only ˜1% over the duration of an iontophoresis experiment (7 hours). As mentioned above, VCV as supplied contained small amounts of ACV (0.5%). However, regeneration of ACV from the prodrug was significantly enhanced at physiological pH (in PBS pH 7.4) as previously reported (Anand and Mitra, 2002) with ˜12% of the prodrug being converted over 7 hrs.

After 4 hours of contact with the dermis, VCV was completely hydrolyzed to ACV and only the latter was detected in the receiver (dermal) compartment, at physiological pH. In contrast, after an equivalent contact period with the SC, enzymatic conversion of VCV was not observed. When ACV was similarly tested, the drug placed in both donor and receptor chambers was stable for the duration of the experiment. Although the data indicate that esterase activity is present in the skin tissue despite prior storage of the skin samples at −20° C. (as previously reported (Steinstrasser and Merkle, 1995), the extent of residual activity was not ascertained in the current investigation; it is of course possible that prodrug conversion in vivo proceeds with even greater efficiency.

Taken together, these preliminary observations supported the practical feasibility of VCV iontophoresis for enhanced delivery of ACV to the deeper skin layers. Since VCV was unaffected by the SC, it was reasonable to anticipate that this cationic prodrug could be iontophoretically delivered to the viable epidermis at enhanced rates and levels compared to the uncharged ACV, before being converted to the active moiety—either in the viable epidermis or in the dermis.

### Example 4

### Passive Topical Drug Delivery

Neither ACV nor VCV could be measured in the receiver after passive delivery from formulations containing 10 mM VCV.HCl in water or 2 mM ACV in 2 mM NaCl. In view of the analytical limit of detection of ACV in the current study (˜0.13 μM), the passive fluxes of ACV and VCV can be considered to be inferior to 1 nmol/cm²/h. This is not surprising considering their polar nature (calculated log P and log D values are −1.76 and −0.78, for ACV and VCV, respectively (Denny, 2003)), and the aqueous formulation used, from which partitioning of these molecules into the lipidic stratum corneum is not favoured. These passive estimates are not dissimilar to the in vitro flux values of ACV observed across human (0.24 nmol/cm²/h) and guinea pig (0.21 nmol/cm²/h) skin from 5% w/w (220 mM; 100-fold higher concentration compared to present work) ACV ointment (Freeman et al., 1986). The topical application of a 220 mM ACV formulation to rabbits in vivo, resulted in dermal concentrations (monitored by microdialysis) that were below the limit of detection (Stagni et al., 2004). Of course, this is not necessarily indicative of poor skin penetration. Indeed, topical formulations are designed to target the medicament to the site of action within the skin tissue and as such employ quantities which are far inferior to those administered systemically for equivalent effect. Drug reaching the highly vascularized dermal papillary layer subsequent to epidermal permeation is rapidly taken up into the general circulation but is difficult to detect by virtue of the rapid and extensive ‘dilution’ of these modest drug amounts. This has been demonstrated, to a certain extent, for commercially available topical ACV formulations (Zovirax®), which contain excipients (such as propylene glycol) to facilitate ACV penetration into the SC. In various in vitro and in vivo model systems, the topical administration of 5% w/w Zovirax® ointment and cream formulations resulted in total epidermal levels that far exceeded therapeutic levels but systemic levels that were analytically non-detectable. However, despite therapeutically acceptable epidermal levels of ACV, model predictions suggest that inadequate antiviral levels in the basal epidermis—the site of HSV type-1 infection, compromise topical therapy. In the experiments described here, ACV delivery was assessed by measurement of receptor levels subsequent to transport of the prodrug or drug across dermatomed porcine ear skin (˜750 μm). Hence, although ACV levels at the site of action were not monitored, receptor concentrations adjacent to the dermis were considered to provide a “ballpark” estimate of those in the basal epidermis in view of previously reported skin distribution profiles for ACV (Parry et al., 1992; Volpato et al., 1998; Yamashita et al., 1993).

### Example 5

### Iontophoretic Drug Transport

Though neither ACV nor VCV could be delivered passively across the skin, application of an iontophoretic current (0.5 mA/cm²) enhanced the percutaneous transport of both these molecules, albeit to different extents. In certain isolated cases, minute amounts of VCV (<2% of ACV levels in receptor) could also be detected and measured in the receiver subsequent to VCV iontophoresis, implying incomplete VCV metabolism. In such cases, both ACV and VCV concentrations were summed to determine the total iontophoretic flux of VCV.

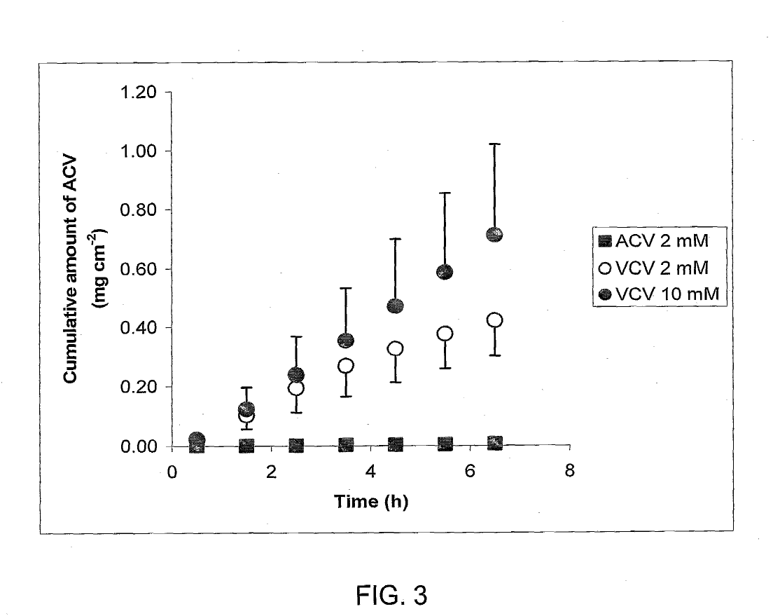

As shown in FIG. 3, receptor levels of ACV were significantly greater subsequent to the iontophoresis of VCV compared to that of ACV, when delivered from equimolar formulations. Nearly 200-fold greater levels of ACV (194+/−82 vs 1.0+/−0.7 μg/cm²) were monitored in the receptor after only 3 hours of current application using a 2 mM solution of the prodrug instead of the parent molecule. The experimental data, therefore, clearly demonstrate that VCV is better suited to iontophoretic delivery relative to its active metabolite, ACV. Moreover, the dissimilar transport kinetics, together with the preliminary hydrolysis assays, indicate that the prodrug is promptly converted to its active metabolite, after passage through the stratum corneum in the vicinity of the epidermal and/or dermal tissues. Since topical delivery of a therapeutic agent results in the establishment of a concentration gradient from the SC towards the subcutaneous tissues, we can expect (all else being equal) ACV concentrations in the receptor to reflect those in the basal epidermis.

Although, at first glance, cumulative ACV transport appeared to be enhanced upon increasing donor VCV concentration from 2 to 10 mM, strict comparison was confounded by donor depletion at the lower applied dose. Indeed, at the lower donor concentration, the amount that had reached the receptor after only 4 hours was equivalent to 50% of the applied dose.

Importantly, the average flux over the first 3 hours of current application (Jt=0-3 h) was not significantly different (p<0.05) for the two formulations (2 mM: 385±158 nmol/cm²/h; 10 mM: 479±258 nmol/cm²/h). Given the virtual absence of a lag-phase and rapid attainment of steady-state kinetics within the same time-frame for the 10 mM formulation (J_{t=0-3 h}=479±258 vs J_{t=0-7 h}=510±219 nmol/cm²/h) the observed Jₜ₌₀₋₃ for the lower concentration was also assumed to approximate the steady-state value. That is to say, in the event of donor replenishment, the iontophoretic transport kinetics for VCV at 2 mM were not expected to differ from those observed for the 10 mM formulation. This non-dependence of flux on donor concentration has been previously reported for the hydrochloride salts of lidocaine, quinine and propranolol when delivered from formulations deficient in competitive charge carriers, e.g., those originating from buffer systems (Marro et al., 2001).

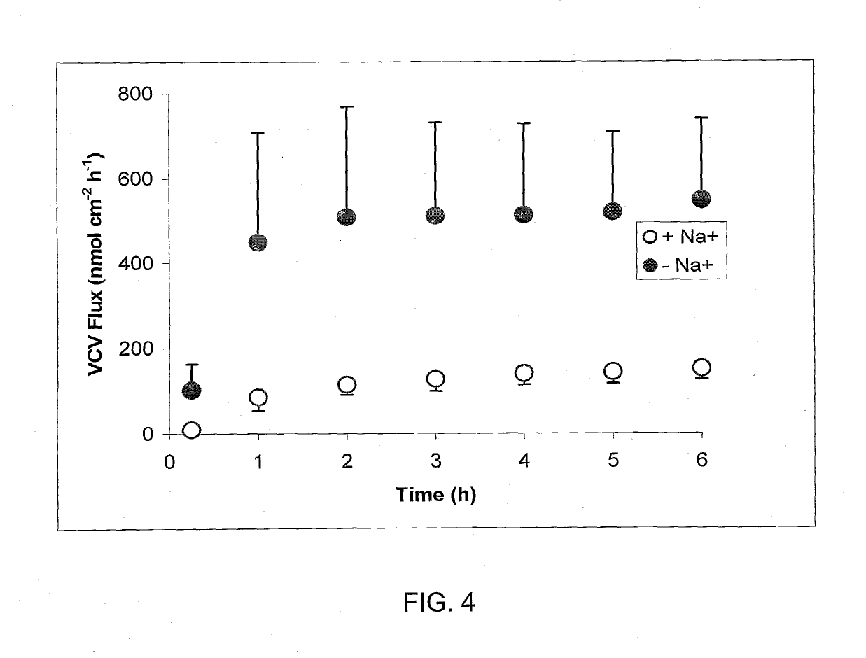

The efficiency of iontophoretic drug transport is influenced by donor electrolyte levels since mobile inorganic ions compete very effectively with higher molecular weight drug molecules to carry the current (Kasting and Keister, 1989). The effect of donor electrolyte levels was evaluated in a series of control experiments conducted without a salt bridge assembly where the anodal/donor compartment contained 10 mM VCV.HCl in PBS pH 7.4 (170.5 mM Na⁺; 1.4 mM K⁺). The resulting fluxes are depicted in FIG. 4, which clearly shows the reduction in transport rates as background electrolyte levels, and hence the number of competing ions, are increased.

The iontophoretic delivery of ACV across human and nude mouse skin has been previously investigated at pH 3.0 (20% ionized) and at pH 7.4 (2% ionized) (Volpato et al., 1998). The donor concentration-normalized fluxes across porcine skin at pH 6.3, in the present study (2.4×10⁻³ cm/h) over 7 hours of iontophoresis compare favourably with those across human skin at physiological pH (2.7×10⁻³ cm/h) (Volpato et al., 1995). In the studies with human skin, the distribution of ACV across various skin layers was also examined; although iontophoresis generally enhanced total skin concentrations of ACV, only formulations at pH 3.0 produced a significant impact on the epidermal levels of the antiviral agent.

### Example 6

### Mechanism of VCV Transport

The iontophoretic flux (J_{TOT}) of a charged species is considered to be the sum of two separate transport processes—electromigration (J_{EM}) and electroosmosis (J_{EO}), assuming negligible passive skin permeability (Sage Jr., 1995):

J_{TOT,VCV}=J_{EM,VCV}+J_{EO,VCV} (equation 2)

Briefly, electromigration occurs as a result of increased molecular transport in the presence of an applied electric field, whereas electroosmosis refers to transport mediated by the bulk solvent flow across the skin (in the anode-to-cathode direction) due to the skin's permselectivity for cations under physiological conditions.

The mechanism governing the iontophoretic transfer of each molecule across the skin was evaluated by virtue of the simultaneous measurement of acetaminophen, a neutral molecule, transported principally by electroosmosis (EO). While ACV was transported across the skin entirely by EO, delivery of the prodrug was due almost entirely to electromigration (EM). The two distinct transport mechanisms for each molecule reflect their ionization state in the respective donor formulations. The unbuffered ACV and VCV formulations had pH values of 6.3 and 5.7, respectively; under these conditions, ACV is effectively uncharged while VCV is fully protonated in the donor phase. Consequently, the transport of uncharged ACV is mediated by the convective solvent movement, whilst that of the protonated VCV is facilitated by electromigration from the anodal electrode. When donor electrolyte levels were increased, the EM contribution to the total flux was reduced from 96 to 73%, due to increased competition from extraneous ions in the formulation to transport charge across the membrane.

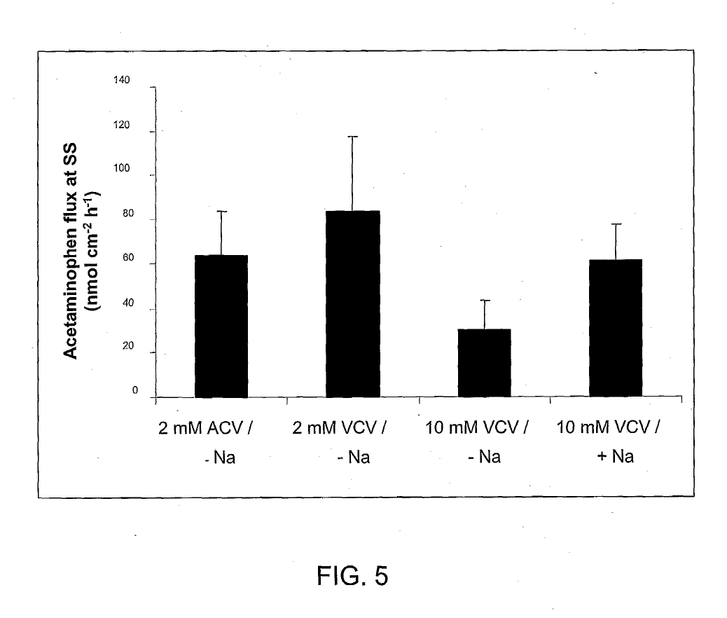

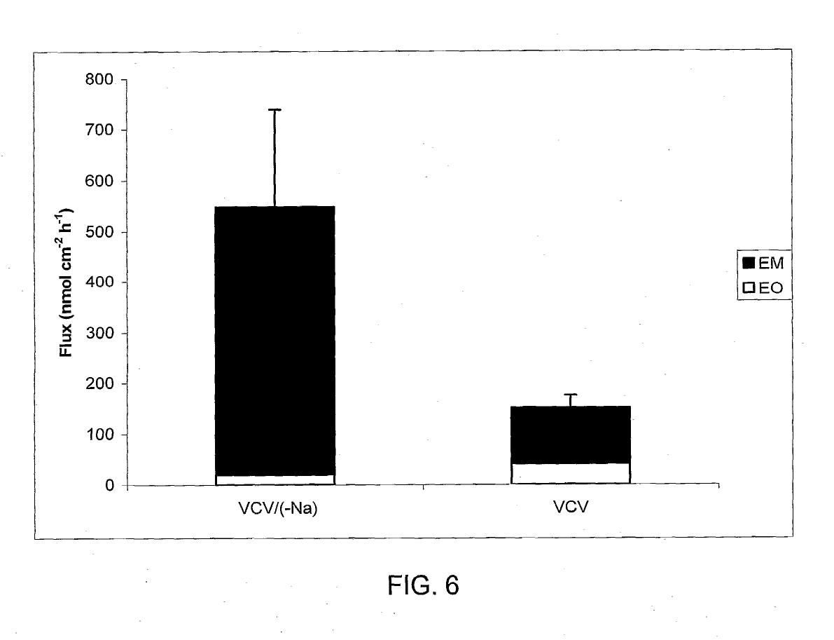

As FIG. 5 shows, in the absence of competing ions (Na⁺), acetaminophen transport (reflecting the EO contribution) was significantly influenced by donor levels of VCV (p<0.05). Basal levels of acetaminophen transport were unaffected by the presence of either 2 mM VCV or ACV (p<0.05). However, when VCV donor concentration was increased from 2 to 10 mM, acetaminophen transport decreased significantly (p<0.05). This suggests that VCV interacts with the skin to reduce convective solvent flow across the membrane, as has been demonstrated previously for other cationic species (Marro et al., 2001). This phenomenon tends to have a negative impact on total iontophoretic transport (e.g., in the presence of background electrolyte, flux may not be linearly proportional to applied dose), and is considered to reduce the benefits of “controlled-delivery” generally offered by iontophoresis. However, for molecules such as VCV, which are overwhelmingly transported by EM (˜95%), the impact of modifying EO on overall transport is virtually imperceptible (FIG. 6).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Patent Application 20050113738
U.S. Patent Application 20050070840
U.S. Patent Application 20040167460
U.S. Patent Application 20040116964
U.S. Patent Application 20040064084
U.S. Patent Application 20040039328
U.S. Patent Application 20030135150
U.S. Patent Application 20030175328
U.S. Patent Application 20030175333
U.S. Pat. No. 6,862,473
U.S. Pat. No. 6,858,018
U.S. Pat. No. 6,629,968
U.S. Pat. No. 6,377,847
U.S. Pat. No. 5,882,677
U.S. Pat. No. 5,738,647
U.S. Pat. No. 6,731,987
U.S. Pat. No. 6,064,908
PCT publication WO 2004062600
Abla et al., Pharm. Res., 22: 2069-2078, 2005.
Abla et al., In: Percutaneous Penetration Enhancers Smith and Maibach (Eds.), CRC Press, Boca Raton, Fla., 177-219, 2006.
Anand et al., J. Pharmacol. Exp. Ther., 311:659-667, 2004.
Anand et al., Pharm. Res., 19:1194-1202, 2002.
Beauchamp et al., Antivir. Chem. Chemother., 3:157-164, 1992.
Boderke et al., J. Invest. Dermatol., 108:83-86, 1997.
Boehnlein et al., Pharm. Res., 11:1155-1159, 1994.
Sage Jr., In: Percutaneous penetration enhancers, Smith and Maibach (Eds.), CRC Press, Boca Raton, Fla., 351-368, 1995.
Denny, J. Biomedicine Biotech., 2003:48-70, 2003.
Dick and Scott, J. Pharm. Pharmacol., 44:640-645, 1992.
Fiddian and Ivanyi, Br. J. Dermatol., 109:321-326, 1983.
Freeman et al., Agents Chemother., 29:730-732, 1986.
Gangarosa and Hill, Int. J. Pharm., 123:159-171, 1995.
Gerscher et al., Br. J. Dermatol., 144:983-990, 2001.
Gerscher et al, Photochem. Photobiol., 72:569-574, 2000.
Jacobson, J. Med. Virol. Suppl., 1:150-153, 1993.
Kalia et al., Adv. Drug Deliv. Rev., 56:619-658, 2004.
Kasting and Keister, J. Control. Release, 8:195-210, 1989.
Laneri et al., Pharm. Res., 16:1818-1824, 1999.
Lopez et al., Photochem. Photobiol., 77:304-308, 2003.
Marro et al., Pharm. Res., 18:1701-1708, 2001.
Martin and Axelrod, Biochim. Biophys. Acta, 26:490-501, 1957.
Parry et al., J. Invest. Dermatol., 98:856-863, 1992.
Piret et al., Antimicrob. Agents Chemother., 44:30-38, 2000.
Potts et al., Pharm. Res., 6:119-124, 1989.
Raborn and Grace, J. Can. Dent. Assoc., 69:498-503, 2003.
Sekkat et al., J. Pharm. Sci., 91:2376-2381, 2002.
Soul-Lawton et al., Antimicrob. Agents Chemother., 39:2759-2764, 1995.
Spruance and Crumpacker, Am. J. Med., 73:315-319, 1982.
Spruance et al., Antimicrob. Agents Chemother., 25:553-555, 1984.
Spruance et al., Antimicrob. Agents Chemother., 25:10-15, 1984.
Spruance et al., J. Infect. Dis., 146:85-90, 1982.
Spruance et al., Antimicrob. Agents Chemother., 30:196-198, 1986.
Spruance et al., Antimicrob. Agents Chemother., 46:2238-2243, 2002.
Stagni et al., Int. J. Pharm., 274:201-211, 2004.
Steinstrasser and Merkle, Pharm. Acta Helv., 70:3-24, 1995.
Stinchicomb et al., Pharm. Res., 13:1519-1523, 1996.
Sung et al., J. Control. Release, 67:1-8, 2000.
Volpato et al., Pharm. Res., 12:1623-1627, 1995.
Volpato et al., J. Control. Release, 50:291-296, 1998.
Yamashita et al., Int. J. Pharm., 89:199-206, 1993.
Yeo and Fiddian, J. Antimicrob. Chemother., 12:95-103, 1983.

1. A method for topical delivery of a protonated antimicrobial compound to an animal comprising the steps of:
a) obtaining a composition comprising molecules of an antimicrobial compound, wherein more than 20% of the molecules would be protonated at pH 7.4;
b) applying the composition to an affected topical area of the animal;
c) subjecting the affected topical area to an electrical current in a manner effective to promote the transport of the compound through the skin of said animal.

2. The method of claim 1, wherein the antimicrobial compound comprises an amino acid ester group.

3. The method of claim 1, wherein the antimicrobial compound is an antiviral, an antifungal compound, or an antibiotic compound.

4. The method of claim 1, wherein the antimicrobial compound is a herpes antiviral.

5-6. (canceled)

7. The method of claim 1, wherein the composition further comprises a buffer, a skin moisturizing agent, a salt, a preservative, or an anesthetic.

8. The method of claim 7, wherein the composition further comprises a buffer, wherein the buffer is a phosphate, carbonate, HEPES or a TRIS buffer.

9-11. (canceled)

12. The method of claim 1, wherein the topical area is the eye, lips, face, skin, genitals, or anus.

13. The method of claim 1, wherein the animal is a human.

14. The method of claim 1, wherein more than 30% of the molecules would be protonated at pH 7.4.

15. (canceled)

16. The method of claim 1, wherein the composition has a pH of about 4.0 to about 8.0.

17-19. (canceled)

20. The method of claim 1, wherein the topical area is a lesion.

21. The method of claim 20, wherein the lesion is caused by a virus, a bacteria, or a fungus.

22-23. (canceled)

24. The method of claim 21, wherein the lesion is a herpes viral lesion.

25. The method of claim 24, wherein the herpes virus lesion is a HSV-1, HSV-2, or VZV lesion.

26. The method of claim 1, wherein the compound is for local delivery or for systemic delivery.

27. (canceled)

28. The method of claim 1, wherein the topical area is about 10 cm² or less.

29. (canceled)

30. The method of claim 1, wherein the electrical current has a current density of about 0.5 mA/cm² or less.

31. (canceled)

32. The method of claim 4, wherein the herpes antiviral compound is valaciclovir.

33. (canceled)

34. The method of claim 7, wherein the composition comprises an anesthetic, wherein the anesthetic is lidocaine, bupivacaine, butacaine, chloroprocaine, cinchocaine, etidocaine, mepivacaine, prilocaine, ropivacaine, or tetracaine.

35. The method of claim 1, wherein the composition is comprised in a patch.

36. The method of claim 35, wherein the patch is clear or comprises a water soluble polymer.

37. (canceled)

38. The method of claim 1, wherein the electrical current is provided by an iontophoresis apparatus.

39. The method of claim 38, wherein the iontophoresis apparatus is a Phoresor II Auto, a Phores PM900, or an Empi Dupel.

40. The method of claim 1, wherein the electrical current is applied for 4 hours or less.

41. (canceled)

42. A kit comprising:
a) a composition comprising molecules of an antimicrobial compound, wherein more than 20% of the molecules would be protonated at pH 7.4;
b) an apparatus capable of supplying an electrical current in a manner effective to promote the transport of the compound through the skin.

43. The kit of claim 42, further comprising instructions for the use of the kit, a wire, or an electrode.

44. (canceled)

45. The kit of claim 43, wherein the kit further comprises an electrode, where the electrode is an anode or a cathode.

46. The kit of claim 42, wherein the kit is comprised in a box.

47. The kit of claim 42, wherein the antimicrobial compound comprises an amino acid ester group.

48. The kit of claim 42, wherein the antimicrobial compound is an antiviral, an antifungal, or an antibiotic.

49. The kit of claim 48, wherein the antimicrobial compound is an antiviral, and wherein the antiviral compound is a herpes antiviral.

50-51. (canceled)

52. The kit of claim 42, wherein the composition further comprises a buffer, a skin moisturizing agent, a salt, a preservative, or an anesthetic.

53. The kit of claim 52, wherein the buffer is a phosphate, carbonate, HEPES or TRIS buffer.

54-56. (canceled)

57. The kit of claim 42, wherein more than 30% of the molecules would be protonated at pH 7.4.

58. (canceled)

59. The kit of claim 42, wherein the composition has a pH of about 4.0 to about 8.0.

60-62. (canceled)

63. The kit of claim 49, wherein the herpes antiviral compound is valaciclovir.

64. The kit of claim 52, wherein the composition further comprises an anesthetic, wherein the anesthetic is lidocaine.

65. (canceled)

66. The kit of claim 42, wherein the composition is comprised in a patch.

67. The kit of claim 66, wherein the patch is clear or comprises a water soluble polymer.

68. (canceled)

69. The kit of claim 42, wherein the apparatus capable of supplying an electrical current in a manner effective to promote the transport of the compound through the skin is an iontophoresis apparatus.

70. The kit of claim 69, wherein the iontophoresis apparatus is a Phoresor II Auto, a Phores PM900, or an Empi Dupel.

71. The kit of claim 66, wherein the patch is about 10 cm² or less.

72-74. (canceled)

75. The method of claim 12, wherein said topical area is the eye.

76. The method of claim 75, wherein said antimicrobial compound is foscarnet, cidofovir, formivirsen, ganciclovir, valganciclovir, trifuridine, vidarabine or idoxuridine.

77. (canceled)

78. The method of claim 35, wherein the patch is formed to the shape of the eye.
